(19) United States
(12) Patent Application Publication
(10) Pub. No.: US 2023/0085499 A1
(43) Pub. Date: Mar. 16, 2023

(54) PHARMACEUTICAL COMPOSITION AND USE THEREOF FOR REPAIRING DAMAGED LIVER, TREATING DISEASE ASSOCIATED WITH DAMAGED LIVER AND IMPROVING FUNCTIONS OF LIVER

(71) Applicant: TAIWAN MITOCHONDRION APPLIED TECHNOLOGY CO., LTD., Zhubei City (TW)

(72) Inventors: Han-Chung CHENG, Zhubei City (TW); Chih-Kai HSU, Zhubei City (TW); Chi-Tang TU, Zhubei City (TW); Shun-Chieh YANG, Zhubei City (TW); An-Ling CHENG, Zhubei City (TW)

(73) Assignee: TAIWAN MITOCHONDRION APPLIED TECHNOLOGY CO., LTD., Zhubei City (TW)

(21) Appl. No.: 17/982,805

(22) Filed: Nov. 8, 2022

Related U.S. Application Data
(63) Continuation-in-part of application No. PCT/MY2021/050039, filed on May 7, 2021.

(30) Foreign Application Priority Data
May 8, 2020 (TW) 109115466

(51) Int. Cl. A61K 35/28; A61P 1/16

(52) U.S. Cl. CPC A61K 35/28; A61P 1/16

(57) ABSTRACT

The composition for improving liver function of the present disclosure includes at least one of mitochondria from autologous cells and exogenous mitochondria, and stem cells. The composition may decrease GOT and GPT indices, indicating the composition of the present disclosure may repair damaged liver and improve the regeneration ability of the damaged liver. The composition of the present disclosure may improve albumin synthesis, prothrombin synthesis and bilirubin metabolism of the liver, indicating the composition of the present disclosure may improve liver's ability of synthesis and metabolism.

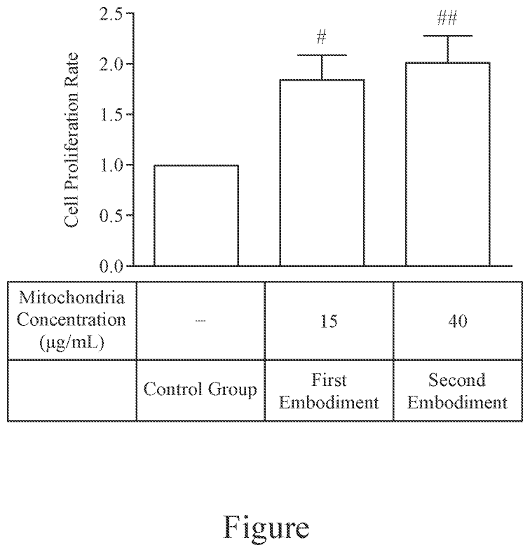

### CROSS-REFERENCE TO RELATED APPLICATIONS

This application is a continuation-in-part of International Application No. PCT/MY2021/050039, filed on May 7, 2021, which claiming priority to Patent Application No(s). 109115466 filed in Republic of China (ROC) on May 8, 2020, the entire contents of which are hereby incorporated by reference.

### BACKGROUND

### 1. Technical Field

This disclosure relates to a composition for improving liver function.

### 2. Related Art

Liver is one of the most important organs in the human body for metabolism. The functions of liver include metabolizing substances such as glucose, lipids, protein, cellulose, hormones and bile, etc. The functions of liver also include synthesizing substances such as protein and coagulation factor, etc., wherein the coagulation factor involves the process of blood coagulation and hematopoiesis. In addition, the functions of liver further include secretion, excretion, bioconversion and detoxication, which are important functions in protecting human body.

The causes for liver dysfunction include infection, damage from alcohol intake, autoimmune problems, genetic, medication and tumors, etc. When liver dysfunction happens, it usually causes inflammation along with liver fibrosis which in turn causes cirrhosis and even loss of liver function. When the liver can't function normally, it will result in abnormal metabolism and central nervous system abnormalities, and thereby affecting the function of other organs, or even becoming life-threating in some sever cases. Currently, many research teams are trying their best to improve liver function and repair liver damage to maintain normal operation of the human body.

### SUMMARY

This disclosure provides a composition including mitochondria and use thereof for improving liver function.

An embodiment of this disclosure provides a use of mitochondria for preparing a composition in repairing liver damage or treating disease associated with liver damage.

An embodiment of this disclosure provides a use of mitochondria for preparing a composition in improving liver function.

An embodiment of this disclosure provides a composition including mitochondria and stem cell. The mitochondria includes at least one of mitochondria extracted from autologous cell and exogenous mitochondria.

The mitochondria for improving liver function of the embodiment of the present disclosure may be used to reduce glutamic-oxalacetic transaminase (GOT) index or glutamic pyruvic transaminase (GPT) index. Accordingly, liver damage may be repaired, liver damage associated disease may be treated, or the regeneration capability of damaged liver may be improved. The composition may also be used to improve protein synthesis, bilirubin metabolism, and prothrombin synthesis. Accordingly, liver functions of synthesis and metabolism may be improved.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. is an experiment result of applying the embodiment of the present disclosure for repairing liver cell damage and improving liver cell proliferation.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

Liver damage usually results in symptoms such as abnormal glutamic-oxalacetic transaminase (GOT) index and glutamic pyruvic transaminase (GPT) index, protein synthesis abnormality, liver cell proliferation abnormality and metabolism abnormality or blood coagulation abnormality. Liver damage includes liver fibrosis, cirrhosis, inflammation, fatty liver disease, alcohol related liver damage, liver resection and liver transplant etc. Mitochondria in liver cells regulates metabolism and balances oxidative stress. Mitochondria abnormality in liver cells is one of the causes for liver disease. In addition, in the liver cells of patients with liver damage, structure abnormality, reduced energy production, and reduced efficiency in redox reaction may also be observed in mitochondria. Therefore, by transplanting healthy mitochondria to the liver, the liver function and the regeneration capacity of damaged liver may be improved, so as to further treat diseases associated with liver damage. Accordingly, the symptoms resulted from liver damaged described above and the effects from liver damage may be alleviated.

An embodiment of this disclosure provides a composition for improving liver function. The composition includes mitochondria and stem cell, and the mitochondria includes at least one of mitochondria extracted from autologous cells and exogenous mitochondria. Mitochondria may be extracted from cells having mitochondria such as monocytes, embryonic stem cells, mesenchymal stem cells, hematopoietic stem cells, CD34+ stem cells, bone marrow stem cells, etc. In some embodiments, mitochondria are preferably obtained from cells of the same species as those to which the composition is applied. For example, human cells are used for extracting mitochondria when the object to be administered the composition is a human; dog cells are used for extracting mitochondria when the object to be administered the composition is a dog. In some embodiments of the present disclosure, mitochondria may be exogenous mitochondria obtained by being taken from cells of the same species as those to which the composition is applied and preserved in vitro or incubated in vitro. In some embodiments of the present disclosure, the composition may further include a pharmaceutically acceptable carrier. The pharmaceutically acceptable carrier includes a carrier used in any standard medical product or cosmetic product, and the carrier may be in semi-solid form or liquid form depending on the form of the composition. For example, the carrier includes, but not limited to, hyaluronic acid, gelatin, emulsifying agent, water, saline solution, buffered saline, ethanol or other substances able to maintain the activity of mitochondria.

In the composition of some embodiments of the present disclosure, the weight of mitochondria may be 1 microgram (m) to 1000 micrograms. In the composition of some embodiments of the present disclosure, the weight of mitochondria may be 5 micrograms to 1000 micrograms. In the composition of some embodiments of the present disclosure, the weight of mitochondria may be 5 micrograms to 200 micrograms. In the composition of the present disclosure, the concentration of mitochondria may be 5 micrograms per milliliter (m/mL) to 400 micrograms per milliliter. In some embodiments of the present disclosure, the concentration of mitochondria may be 25 micrograms per milliliter to 400 micrograms per milliliter. In some embodiments of the present disclosure, the concentration of mitochondria may be 75 micrograms per milliliter to 200 micrograms per milliliter. In another part of the embodiments of the present disclosure, the concentration of mitochondria may be 5 micrograms per milliliter to 80 micrograms per milliliter. In yet another part of the embodiments of the present disclosure, the concentration of mitochondria may be 15 micrograms per milliliter to 40 micrograms per milliliter.

The composition of the present disclosure may be administered to the liver through oral, injection or other methods. In some embodiments of the present disclosure, the composition is preferably administered to the liver directly by injection, but the present disclosure is not limited thereto. In some embodiments of the present disclosure, the effective dose of mitochondria in the composition is 0.5 micrograms per gram of liver to 1000 micrograms per gram of liver. The effective dose of mitochondria in the composition of some embodiments of the disclosure is 0.5 micrograms per gram of liver to 100 micrograms per gram of liver. The effective dose of mitochondria in the composition of some embodiments of the disclosure is 1 microgram per gram of liver to 4.5 micrograms per gram of liver.

In some embodiments of the present disclosure, the composition further includes stem cells. Stem cells may secrete a plurality of kinds of growth factors, the growth factors may improve the repair ability of a variety of cells. Stem cells are, for example, embryonic stem cell, mesenchymal stem cell, hematopoietic stem cell, CD34+ stem cell, bone marrow stem cell. In some embodiments of the present disclosure, a ratio of the weight of mitochondria to the number of stem cells is 1 microgram to 3.3×10⁴˜1×10⁵.

In some embodiments of the present disclosure, the composition comprising mitochondria may effectively improve the proliferation of cells, which means the damaged liver is repaired and the proliferation of the liver is improved. Further, as the concentration of mitochondria increases, the repair efficiency is better.

In some embodiments of the present disclosure, the composition including mitochondria may reduce GOT index or GPT index, which indicates that the composition including mitochondria of the present disclosure may effectively repair the damaged liver. The composition including mitochondria may increase the concentration of albumin, which indicates that the composition including mitochondria of the present disclosure may effectively improve liver's ability to synthesize albumin. composition including mitochondria may shorten the prothrombin time, which indicates that the composition including mitochondria of the present disclosure may effectively improve liver's ability of synthesizing prothrombin. Furthermore, the composition including mitochondria and human adipose derived stem cell shows a better improvement in the biochemical indices compared to the composition containing only human adipose stem cells or only mitochondria, which indicates that the use of human adipose stem cells and mitochondria together has synergistic effect.

In some embodiments of the present disclosure, the composition comprising mitochondria may treat diseases associated with liver damage, wherein liver damage includes liver fibrosis, cirrhosis, inflammation, fatty liver disease, alcohol related liver damage, liver resection and liver transplant etc.

The following describes how to prepare the composition of the embodiments of the present disclosure.

[Mitochondria Extraction]

The mitochondria used in the embodiments of the present disclosure are extracted from human adipose derived stem cells (ADSC). The ADSCs are incubated in a cell culture dish until the number of the ADSCs reaches 1.5×10⁸, and rinsed with Dulbecco's phosphate-buffered saline (DPBS). Then, after DPBS is removed, trypsin is added for cell detachment.

After the cells react for 3 mins under 37° C., stem cell culture medium (Keratinocyte SFM (1X) solution (Gibco), bovine pituitary extract (BPE, Gibco), 10% (weight percentage concentration) of fetal bovine serum (HyClone)) is added to stop the reaction. Then, the cells are washed and then dispersed, and centrifuged at 600 g for 10 minutes, and the supernatant is removed. Next, the cells are added with 80 ml of IBC-1 buffer (225 mM mannitol, 75 mM sucrose, 0.1 mM EDTA, 30 mM Tris-HCl pH 7.4), and then grinded for 15 times on ice in a homogenizer. Then, the homogenized mixture is centrifuged at 1000 g for 15 minutes, the supernatant is collected into another centrifuge tube, and then the remaining mixture is centrifuged at 9000 g for another 10 minutes to remove last supernatant. The precipitate obtained finally is the mitochondria. The mitochondrial precipitate is added with 1.5 ml of IBC-2 buffer (225 mM mannitol, 75 mM sucrose, 30 mM Tris-HCl pH 7.4) and proteolytic enzyme inhibitor, and stored at 4° C.

### [Experiment 1] Repairing Damage of Liver Cells

In this experiment, HepG2 cell line is selected as a cell model for evaluating liver damage. The HepG2 cell line is a hepatocarcinoma cell line isolated and established from primary hepatic embryonic cell tumor. Since HepG2's metabolic enzymes features complete biotransformation characteristics, high similarity to normal human liver cells, and HepG2's metabolic enzymes do not change with cell culture generation, HepG2 cell line is often used as an ideal cell model for studying liver cell metabolism and liver cell damage. The cell culture generation of HepG2 used in this experiment is from the 4th generation to the 10th generation. HepG2 cell culture medium includes DMEM medium and 10% (weight percentage concentration) fetal bovine serum.

D-galactosamine is a substance often used in inducing liver cell damage. D-galactosamine is often used in inducing the cell damage or apoptosis, in order to evaluate the therapeutic effect on liver damage and to further use as a treatment evaluation model. This experiment uses D-galactosamine as a substance inducing HepG2 cell damage.

Alamar blue is a detection reagent used to detect cell viability. The resazurin in the detection kit is a non-toxic, dark blue dye redox indicator that can penetrate cell membranes and has low fluorescence. When resazurin enters healthy cells, it will be reduced to pink and highly fluorescent resorufin due to the reducing environment in living cells. The higher the light absorption or fluorescence value of resorufin, the higher the cell viability. A higher cell viability indicates healthier cells are and greater proliferation ability. Greater cell proliferation ability indicates a large number of cells. Therefore, Alma blue may be used as an indicator of cytotoxicity to determine cell survival rate and cell proliferation rate.

In this experiment, D-galactosamine is used to induce HepG2 cell damage, and the composition including mitochondria of each embodiments is administered to the liver to repair cell damage and improve cell proliferation. Alma blue detection reagent is used to evaluate the effects of repairing liver cell damage by using the composition including mitochondria, and is represented by cell proliferation rate. The following describes the experiment process.

First, the experiment is conducted by using HepG2 cells with cell culture ranging between 4th generation to 10th generation. When HepG2 cells are cultured to take up 80% of the culture dish, the culture medium is removed, and the cells are rinsed with phosphate buffered saline (PBS). Then, 0.25% trypsin is added to react with the cells at 37° C. for 5 minutes, and then HepG2 cell culture medium is added to stop the reaction. Next, the mixture is centrifuged at 1000 revolutions per minute (rpm) for 5 minutes to remove supernatant, and new HepG2 cell culture medium is added for cell count.

Then, HepG2 cells are cultured in a 24-well plate for 24 hours with 5×10⁴ HepG2 cells in each well. Then, D-galactosamine is added to the 24-well plate with a concentration of 25 mM in each well. After the cells have been treated with D-galactosamine for 4 hours, the composition including mitochondria of the embodiment is added, and the HepG2 cells in the 24-well plate are cultured with the composition including mitochondria for 20 hours. After the culture is completed, the HepG2 cells are washed by phosphate buffer, and the culture medium is replaced with a culture medium containing Alma blue. The HepG2 cells are cultured in the medium containing Alma blue for 3 hours. After the culture is completed, the fluorescence is measured at the wavelength of OD530/595 to determine the proliferation rate of HepG2 cells.

The result of the experiment is as shown in Table 1 and FIG. FIG. is an experiment result of applying the embodiment of the present disclosure for repairing liver cell damage and improving liver cell proliferation. The control group includes HepG2 cells which have been treated with D-galactosamine and are not cultured with mitochondria, such that the control group represents damaged cells. The vertical axis is the rate of cell proliferation relative to the control group, which is referred to herein as the cell proliferation rate. The concentration of mitochondria is the concentration of mitochondria in the disk. The experimental result shows that both the composition of first embodiment (mitochondria at a concentration of 15 μg/mL) and the composition of second embodiment (mitochondria at a concentration of 40 μg/mL) may increase the cell proliferation rate of damaged HepG2 cells. Compared with the control group, the cell proliferation rate in the first embodiment is significantly increased (#: p<0.05). Compared with the control group, the cell proliferation rate in the second embodiment is increased more significantly (#: p<0.01).

TABLE 1

| | Mitochondrial | Cell Proliferation |
|---|---|---|
| | Concentration | Rate Relative |
| | (μg/mL) | to Control Group |
| Control Group | — | 1 |
| First Embodiment | 15 | 1.85 |
| Second Embodiment | 40 | 2.01 |

### [Experiment 2] Improving Liver Function

This experiment uses 8-week-old male Wistar rat as the animal model for evaluating liver damage.

Thioacetamide (TAA) is a substance commonly used to induce liver damage in animal models. Thiacetamide was injected into the peritoneal cavity of the 8-week-old male Weiss rats at a dose of 200 mg/kg, once every three days for a total of 20 times (60 days in total). The liver of Weiss rats treated under this condition will be damaged. This model is often used as an evaluation model for liver fibrosis or liver damage in animals.

The liver function is impaired, and the biochemical index in the blood will also change. Glutamic-oxalacetic transaminase (GOT), glutamic pyruvic transaminase (GPT), albumin, prothrombin time and total bile are all biochemical indices commonly used to evaluate liver function.

Glutamic-oxalacetic transaminase (GOT) and glutamic pyruvic transaminase (GPT) mainly exist in liver cells. The two enzymes are released into blood due to the damage of liver cells by drugs, alcohol or viruses, causing the increase of both glutamic-oxalacetic transaminase (GOT) index and glutamic pyruvic transaminase (GPT) index. Therefore, by measuring the concentration of these two enzymes in the blood, the degree of liver cell damage may be determined. Therefore, GOT and GPT may be used as indices for evaluating liver function.

Albumin is produced by the liver and is the most abundant protein in human plasma. A decrease of albumin in the blood may be caused by hepatitis and cirrhosis. Therefore, the concentration of albumin in the blood may be used as one of the indices for evaluating liver synthesis function.

A variety of coagulation factors are also produced by the liver. When the production capability of the liver decreases, the coagulation factors also decreases, resulting in prolonged clotting time. The prothrombin time is the time required for the conversion of prothrombin into thrombin after adding excessive amount of tissue factor to the platelet-deficient plasma to coagulate the plasma. Therefore, prothrombin time may be used as one of the indices for evaluating liver synthesis function.

Bilirubin in the blood can be categorized into direct bilirubin and indirect bilirubin, and the combination of the two is called total bilirubin. Bilirubin is a product generated by destruction of red blood cells and is absorbed and metabolized by the liver. When the liver's ability to metabolize bilirubin decreases, bilirubin flows into the blood. Therefore, the total bilirubin in the blood may be used as one of the indices for evaluating liver metabolic function.

Tests on glutamic-oxalacetic transaminase (GOT), glutamic pyruvic transaminase (GPT), albumin and total bilirubin are described as follows. The blood samples collected from the rats are left at room temperature for about 1 hour to coagulate. The samples are centrifuged by a refrigerated centrifuge at 14000 g, 4° C. for 5 minutes to separate the serum. An automated clinical chemistry analyzer (Fujifilm, DRI-CHEM 4000i) is used to measure glutamic-oxalacetic transaminase (GOT), glutamic pyruvic transaminase (GPT), albumin and total bilirubin.

The detection of prothrombin time is as follows. The blood samples collected from the rat are mixed with an anticoagulant containing citric acid. The mixture is centrifuged at 3000 g for 10 minutes, and the serum is collected. 100 microliters of serum is mixed and reacted with 200 microliters of thromboplastin (Neoplastin CI, Diagnostica Stago). Biomerieux Option 2 Plus Coagulation Analyzer (Behnk Electronick, Norderstedt, Germany) is used for blood clotting time analysis.

In this experiment, thioacetamide is used to induce liver damage in Weiss rats under the conditions of the above-mentioned evaluation mode, and the composition including mitochondria of the embodiment is administered to the liver to improve the function of the damaged liver. Biochemical indices, such as glutamic-oxalacetic transaminase (GOT), glutamic pyruvic transaminase (GPT), albumin, total bilirubin and prothrombin time, are measured to evaluate the effects of the composition including mitochondria on the improvement of liver functions. The experiment procedure is explained below.

First, thioacetamide is injected into the peritoneal cavity of several 8-week-old male Weiss rats at a dose of 200 mg/kg, once every three days for a total of 20 times (60 days in total). Blood samples are collected from the hearts of the rats with induced liver damage, and the blood samples are analyzed for biochemical index. The TAA group is consisted of rats with liver damage and has been treated with thioacetamide. The control group is consisted of normal rats has not been treated with thioacetamide.

The experiment result is as shown in Table 2. The result shows the biochemical index differences between rats with damaged liver and normal rats. Increases in glutamic-oxalacetic transaminase (GOT) index and glutamic pyruvic transaminase (GPT) index indicates damaged liver. A decrease in albumin indicates that the liver's ability to synthesize albumin is impaired. An increase in prothrombin time indicates that the liver's ability to synthesize coagulation factors is impaired. An increase in total bilirubin indicates that the liver's ability to metabolize bilirubin is impaired.

TABLE 2

| | Control Group | TAA Group |
|---|---|---|
| GOT (unit/liter) | 88 ± 28 | 567 ± 53 |
| GPT (unit/liter) | 43 ± 13 | 146 ± 42 |
| Albumin (g/dL) | 4.2 ± 0.5 | 2.3 ± 0.5 |
| Prothrombin Time (sec) | 10.13 ± 0.23 | 17.2 ± 2.3 |
| Total Bilirubin (mg/dL) | 0.028 ± 0.04 | 0.0053 ± 0.008 |

Then, repeat the above steps to induce liver damage in rats. The injection of the control group and the injections including mitochondria of third to five embodiments are injected into rat liver. In detail, in the control group, 0.2 ml of saline solution is used as injection only. The experimental group includes: a group in which 1×10⁶ human adipose stem cells are prepared within 0.2 ml of saline solution as injection, and the concentration of human adipose stem cells is 5×10⁶ per milliliter; a third embodiment in which 15 μg of mitochondria is prepared within 0.2 ml of saline solution as injection, and the concentration of mitochondria is 75 micrograms per milliliter (m/mL); a fourth embodiment in which 40 μg of mitochondria is prepared within 0.2 ml of saline solution as injection, and the concentration of mitochondria is 200 μg/mL; and a fifth embodiment in which 15 μg of mitochondria and 1×10⁶ human adipose stem cells are prepared within 0.2 ml of saline solution as injection, wherein the concentration of human adipose stem cells is 5×10⁶ per milliliter, the concentration of mitochondria is 75 μg/mL, and the ratio of the weight of mitochondria to the number of stem cells is 1 microgram to 6.7×10⁴. The above-mentioned injection is directly injected into the damaged liver by multi-point injection for treatment. The blood samples are collected from the heart of the rats on the 14th day after the injection treatment, and the blood samples were analyzed for biochemical index. The weight of the liver of the rats in this experiment is 9.7 to 9.9 grams, and the effective dose of mitochondria in the composition of the embodiment of the present disclosure used is 0.5 micrograms to 100 micrograms per gram of liver, but the present disclosure is not limited thereto. In some embodiments of the present disclosure, the effective dose of mitochondria in the composition of the embodiment of the present disclosure used is 0.5 micrograms to 1000 micrograms per gram of liver. In some embodiments of the present disclosure, the effective dose of mitochondria in the composition of the embodiment of the present disclosure used is 1.5 micrograms to 4.1 micrograms per gram of liver.

Please refer to Table 3 for the experimental results. The experimental results show that the administration of human adipose stem cells, mitochondria and their combination to rats may reduce glutamic-oxalacetic transaminase (GOT) index and glutamic pyruvic transaminase (GPT) index, increase albumin concentration, reduce prothrombin time, and reduce total bilirubin. In addition, according to the experimental result of the fifth embodiment, the composition including human adipose stem cells and mitochondria has better improvement in each biochemical index than the composition comprising only human adipose stem cells or only mitochondria.

TABLE 3

| | Control | | 3rd | 4th | 5th |
|---|---|---|---|---|---|
| | Group | ADSC | embodiment | embodiment | embodiment |
| Concentration | — | 5 × 10⁶ | 75 μg/mL | 200 μg/mL | 75 μg/mL |
| | | cells/mL | mitochondria | mitochondria | mitochondria + |
| | | ADSC | | | 5 × 10⁶ cells/mL |
| | | | | | ADSC |
| Dosage (mL) | 0.2 | 0.2 | 0.2 | 0.2 | 0.2 |
| GOT (unit/liter) | 302 ± 24 | 204 ± 13 | 193 ± 23 | 167 ± 19 | 163 ± 17 |
| GPT (unit/liter) | 103 ± 22 | 92 ± 14 | 86 ± 13 | 67 ± 11 | 63 ± 15 |
| Albumin (g/dL) | 2.7 ± 0.16 | 3.21 ± 0.05 | 3.3 ± 0.04 | 3.63 ± 0.09 | 3.58 ± 0.12 |
| Prothrombin | 15.2 ± 1.3 | 13.14 ± 0.57 | 12.88 ± 0.43 | 10.46 ± 0.36 | 10.88 ± 0.67 |
| Time (sec) | | | | | |
| Total Bilirubin | 0.049 ± 0.03 | 0.041 ± 0.02 | 0.039 ± 0.02 | 0.031 ± 0.03 | 0.032 ± 0.02 |
| (mg/dL) | | | | | |

In view of the above description, the composition for improving liver function provided by the present disclosure may be used to reduce glutamic-oxalacetic transaminase (GOT) index and glutamic pyruvic transaminase (GPT) index. The reduction of GOT and GPT indices indicates that the composition is capable of repairing liver damage, treating liver damage-associated diseases, or improving the regeneration ability of the damaged liver. The composition may be used to improve the liver's ability to synthesize albumin, metabolize bilirubin, and synthesize prothrombin. The improvement of albumin synthesis, bilirubin metabolism and prothrombin synthesis indicates that the composition may improve the liver's ability for synthesis and metabolism.

What is claimed is:

1. A use of mitochondria for preparing a composition in repairing liver damage or treating disease associated with liver damage, wherein the composition comprises at least one of mitochondria extracted from autologous cell and exogenous mitochondria, and a stem cell, and the mitochondria are out of the stem cell.

2. The use according to claim 1, wherein said repairing liver damage comprises: reducing glutamic-oxalacetic transaminase (GOT) index; reducing glutamic pyruvic transaminase (GPT) index; or improving regeneration capability of damaged liver.

3. The use according to claim 1, wherein said liver damage comprises liver fibrosis, cirrhosis, inflammation, fatty liver disease, alcohol related liver damage, liver resection or liver transplant.

4. A use of mitochondria for preparing a composition in improving protein synthesis ability and metabolism ability of liver.

5. The use according to claim 4, wherein said improving protein synthesis ability comprises improving ability of liver to synthesize albumin or prothrombin.

6. The use according to claim 4, wherein said improving protein metabolism ability comprises improving ability of liver to metabolize bilirubin.

7. The use according to claim 1, wherein the composition is administered to liver through injection, and an effective dose of mitochondria in the composition is 0.5 micrograms per gram of liver to 1000 micrograms per gram of liver.

8. The use according to claim 4, wherein the composition is administered to liver through injection, and an effective dose of mitochondria in the composition is 0.5 micrograms per gram of liver to 1000 micrograms per gram of liver.

9. A composition, comprising:
at least one of mitochondria extracted from autologous cell and exogenous mitochondria; and
a stem cell,
wherein the mitochondria are out of the stem cell.

10. The composition according to claim 9, wherein a weight of the mitochondria is 1 microgram to 1000 micrograms.

11. The composition according to claim 9, wherein a ratio of a weight of the mitochondria to a number of the stem cells is 1 microgram to 3.3×10⁴˜1×10⁵.

12. The composition according to claim 9, wherein the composition is used for improving liver function.
